(19) United States
(12) Patent Application Publication
(10) Pub. No.: US 2007/0167758 A1
(43) Pub. Date: Jul. 19, 2007

(54) AUTOMATED DETECTION OF CARDIAC MOTION USING CONTRAST MARKERS

(76) Inventor: BENEDICT JAMES COSTELLO, Berkeley, CA (US)

Correspondence Address: BOZICEVIC, FIELD & FRANCIS LLP (PRTS);(PROTEUS BIOMEDICAL,INC), 1900 UNIVERSITY AVENUE, SUITE 200, EAST PALO ALTO, CA, 94303, US

(21) Appl. No.: 11/562,911

(22) Filed: Nov. 22, 2006

Related U.S. Application Data
(60) Provisional application No. 60/739,393, filed on Nov. 23, 2005.

(51) Int. Cl. A61B 8/00

(52) U.S. Cl. 600/437

(57) ABSTRACT

Techniques are provided for automatically detecting cardiac motion using contrast markers. The contrast markers can be located in images generated by an external imaging device. The contrast markers can be used for locating regions of interest in a heart and their relative motion. The techniques for determining cardiac motion are objective, automated, and reproducible from session to session. Techniques are also provided for automatically analyzing cardiac motion using contrast markers and outputting data or commands that can be used to facilitate or to direct cardiac therapies. The cardiac motion data can be provided in a therapeutically useful output format, such as waveforms, text, and graphics. As another example, a feedback system can analyze the cardiac motion data and can generate feedback commands that cause a cardiac motion regulating device to automatically adjust heart motion in real-time.

### CROSS-REFERENCE TO RELATED APPLICATIONS

This application claims priority to: U.S. Provisional Application Ser. No. 60/739,393 filed on Nov. 23, 2005; the disclosure of which priority application is herein incorporated by reference.

### INTRODUCTION

1. Field of the Invention

The present invention relates to techniques for automating cardiac motion detection, and more particularly, to techniques for automatically detecting cardiac motion by locating contrast markers in images generated by an external imaging device.

2. Background

In a diverse array of applications, the evaluation of tissue motion is desirable, e.g., for diagnostic or therapeutic purposes. An example of an application that requires an evaluation of tissue motion is cardiac resynchronization therapy (CRT). In CRT, cardiac tissue motion is observed by traditional ultrasound techniques.

CRT is an important new medical intervention for patients suffering from heart failure, e.g., congestive heart failure (CHF). When congestive heart failure occurs, symptoms develop due to the heart's inability to function properly. Congestive heart failure is characterized by gradual decline in cardiac function punctuated by severe exacerbations leading eventually to death. It is estimated that over five million patients in the United States suffer from this malady.

The aim of resynchronization pacing is to induce the interventricular septum and the left ventricular free wall of the heart to contract at approximately the same time. Resynchronization therapy seeks to provide a contraction time sequence that will most effectively produce maximal cardiac output with minimal total energy expenditure by the heart. The optimal timing is calculated by reference to hemodynamic parameters such as dP/dt, the first time-derivative of the pressure waveform in the left ventricle. The dP/dt parameter is a well-documented proxy for left ventricular contractility.

In current practice, external ultrasound measurements are used to calculate dP/dt. Such external ultrasound is used to observe wall motion directly. Most commonly, the ultrasound operator uses the ultrasound system in a tissue Doppler mode, a feature known as tissue Doppler imaging (TDI), to evaluate the time course of displacement of the septum relative to the left ventricle free wall. The current view of clinicians is that ultrasonographic evaluation using TDI or a similar approach may become an important part of qualifying patients for CRT therapy.

As currently delivered, CRT therapy is effective in about half to two-thirds of patients implanted with a resynchronization device. In approximately one-third of these patients, this therapy provides a two-class improvement in patient symptoms as measured by the New York Heart Association scale. In about one-third of these patients, a one-class improvement in cardiovascular symptoms is accomplished. In the remaining third of patients, there is no improvement or, in a small minority, a deterioration in cardiac performance. This group of patients is referred to as non-responders. It is possible that the one-class New York Heart Association responders are actually marginal or partial responders to the therapy, given the dramatic results seen in a minority.

The synchronization therapy, in order to be optimal, targets the cardiac wall segment point of maximal delay, and advances the timing to synchronize contraction with an earlier contracting region of the heart, typically the septum. However, the current placement technique for CRT devices is usually empiric. A physician will cannulate a vein that appears to be in the region described by the literature as most effective. The device is then positioned, stimulation is carried out, and the lack of extra-cardiac stimulation, such as diaphragmatic pacing, is confirmed. With the currently available techniques, rarely is there time or means for optimizing cardiac performance.

When attempted today, clinical CRT optimization must be performed by the laborious manual method of an ultrasonographer evaluating cardiac wall motion at different lead positions and different interventricular delay (IVD) settings. The IVD is the ability of pacemakers to be set up with different timing on the pacing pulse that goes to the right ventricle versus the left ventricle. In addition, all pacemakers have the ability to vary the atrio-ventricular delay, which is the delay between stimulation of the atria and the ventricle or ventricles themselves. These settings and the location of the left ventricular stimulating electrode itself can be important in resynchronizing the patient.

There is currently no useful clinically available means of determining optimal CRT settings on a substantially automatic, real-time, machine-readable basis. It would be an important advancement in cardiology to have an objective means for monitoring cardiac motion in real-time for setting the functions of cardiac resynchronization therapy pacemakers, with further application to the pharmacologic management of heart failure patients, arrhythmia detection and ischemia detection, etc.

### SUMMARY

The present invention provides techniques for automatically determining cardiac motion by detecting contrast markers in images generated by an external imaging device. The contrast markers can be used for locating regions of interest in a heart and determining cardiac motion for the purpose of providing cardiac therapies. The present invention provides techniques for determining cardiac motion that are objective, automated, and reproducible from session to session.

The present invention also provides techniques for automatically analyzing cardiac motion using contrast markers and outputting data or commands that can be used to facilitate or to direct cardiac therapies. Cardiac motion data can be provided in a variety of therapeutically useful output formats, such as waveforms, text, and graphics. According to one embodiment, a feedback system can analyze cardiac motion data and generate feedback commands that cause a cardiac motion regulating device to automatically adjust cardiac motion in real-time.

Also provided are systems and related products, e.g., computer programming, which find use in practicing embodiments of the inventive methods described herein. The methods described herein find use a variety of different applications, some of which are reviewed below in greater detail.

### BRIEF DESCRIPTION OF THE FIGURES

FIG. 1 illustrates an electrical tomography system with contrast markers that are embedded in leads, according to an embodiment of the present invention.

FIGS. 2A-2E illustrate specific examples of contrast markers embedded in leads of a tomography device, according to various embodiments of the present invention.

FIG. 3 illustrates stand-alone contrast markers attached to cardiac tissue, according to another embodiment of the present invention.

FIGS. 4A-4C illustrate external imaging devices that can generate images of contrast markers in cardiac tissue, according to various embodiments of the present invention.

FIG. 5 illustrates a process for locating contrast markers and outputting cardiac motion data, according to an embodiment of the present invention.

FIG. 6 illustrates a process for analyzing cardiac motion from contrast markers and providing feedback commands that control the regulation of cardiac motion, according to another embodiment of the present invention.

FIG. 7 illustrates an example of a computing system that can be used to implement various embodiments of the present invention.

### DETAILED DESCRIPTION

According to embodiments of the present invention, contrast markers are placed in a patient's heart for the purpose of providing cardiac therapies (e.g., CRT) and/or diagnoses. The contrast markers are objects that can be identified in images (e.g., video images) generated by an imaging device, such as an external imaging device. Systems and methods of the present invention can use the images generated by the imaging device to unambiguously determine the location of the contrast markers and their relative motion. In those embodiments where an external imaging device is used to generate images of the contrast markers, the present invention provides short-term measurement of cardiac motion, e.g., in a hospital or in a doctor's office.

In contrast to existing methods for measuring heart synchronicity, the markers can be identified by computerized image recognition software. Once the markers are located in the images, additional information can be extracted from the images, such as the motion of the markers and the relative motion of the markers with respect to each other. The relative motion of the markers can be analyzed for a variety of different purposes, e.g., to compute cardiac synchronicity and contractility, or to detect ventricular dyssynchrony. Output data can be generated in variety of formats, including waveforms representing cardiac motion, text, or graphics.

Contrast markers of the present invention can be used to analyze cardiac motion and assist a clinician in making therapeutic decisions. For example, the cardiac motion output data can be used to adjust a pacemaker's IVD or atrio-ventricular delay settings to improve cardiac synchronization. The present invention provides techniques for locating regions of interest in a heart and their relative motion that are objective, automated, and reproducible from session to session.

In the subject methods, data from one or more contrast markers stably associated with the tissue location of interest, e.g. a cardiac location, is detected to evaluate movement of the tissue location. In certain embodiments, location data for a given marker is detected at least twice over a duration of time, e.g., to determine whether the marker has moved or not over the period of time, and therefore whether or not the tissue location of interest has moved over the period of time of interest. In certain embodiments, a change in location of the marker is detected to evaluate movement of the tissue location, e.g. a method of determining cardiac wall motion.

By “stably associated with” is meant that the marker element is substantially if not completely fixed relative to the tissue location of interest such that when the tissue location of interest moves, the marker element also moves. As the marker is stably associated with the tissue location, its movement is at least a proxy for, and in certain embodiments is the same as, the movement of the tissue location to which it is stably associated, such that movement of the sensing element can be used to evaluate movement of the tissue location of interest. The marker element may be stably associated with the tissue location using any convenient approach, such as by attaching the marker element to the tissue location by using an attachment element, such as a hook, etc., by having the marker element on a structure that compresses the marker element against the tissue location such that the two are stably associated, etc.

In certain embodiments, a single marker element is employed. In such methods, evaluation may include monitoring movement of the tissue location over a given period of time. In certain embodiments, two or more distinct marker elements are employed to evaluate movement of two or more distinct tissue locations. The number of different marker elements that are employed in a given embodiment may vary greatly, where in certain embodiments the number employed is 2 or more, such as 3 or more, 4 or more, 5 or more, 8 or more, 10 or more, etc. In such multi-marker element embodiments, the methods may include evaluating movement of the two or more distinct locations relative to each other.

In certain embodiments, the subject methods include providing a system that includes: (a) an imaging element to generate images of the contrast markers; and (b) one or more marker elements that are stably associated with the tissue location of interest. This providing step may include either implanting one or more new elements into a body, or simply employing an already existing implanted system, e.g., a pacing system, etc. This step, if employed, may be carried out using any convenient protocol, where a variety of protocols are well known to those of skill in the art.

The subject methods may be used in a variety of different kinds of animals, where the animals are typically “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g., rabbits) and primates (e.g., humans, chimpanzees, and monkeys). In many embodiments, the subjects or patients will be humans.

The tissue movement evaluation data obtained using the subject methods may be employed in a variety of different applications, including but not limited to monitoring applications, treatment applications, etc. Certain applications in which the data obtained from the subject methods finds use are further reviewed in greater detail below.

Specific System and Method Embodiments

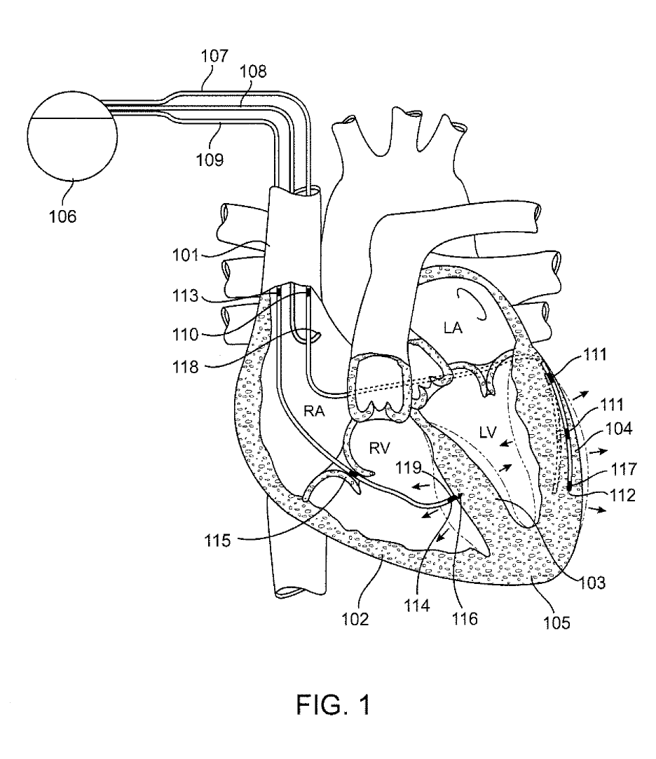

FIG. 1 provides a four chamber view of the heart with an example of a tomography device (e.g., a cardiac timing device) having leads that contain contrast markers, according to an embodiment of the present invention. The tomography device includes a pacemaker 106, a right ventricle electrode lead 109, a right atrium lead 108, and a left ventricle cardiac vein lead 107. The leads 107, 108 and 109 contain contrast markers that appear in images generated by an external imaging device. The leads typically have a small diameter, e.g., 1-2 mm. Also shown in FIG. 1 are the right ventricle lateral wall 102, interventricular septal wall 103, apex of the heart 105, and a cardiac vein on the left ventricle lateral wall 104.

The left ventricle electrode lead 107 is comprised of a lead body and one or more electrodes 110, 111 and 112. The distal electrodes 111 and 112 can be located in the left ventricle cardiac vein to provide regional contractile information about this region of the heart. The distal end 117 of lead 107 can be fixed to the left ventricle free wall 104. The most proximal electrode 110 is located in the superior vena cava in the base of the heart. This basal heart location is essentially unmoving and therefore can be used as one of the fixed reference points for the cardiac wall motion sensing system.

In one embodiment, electrode lead 107 can be constructed with standard materials for a cardiac lead, such as silicone or polyurethane for the lead body, and MP35N for the coiled or stranded conductors connected to Pt—Ir (90% platinum, 10% iridium) electrodes 110, 111, and 112. Alternatively, these device components can be connected by a multiplex system (e.g., as described in published United States Patent Application publication nos.: 20040254483 titled “Methods and systems for measuring cardiac parameters”; 20040220637 titled “Method and apparatus for enhancing cardiac pacing”; 20040215049 titled “Method and system for remote hemodynamic monitoring”; and 20040193021 titled “Method and system for monitoring and treating hemodynamic parameters”; the disclosures of which are incorporated by reference herein), to the proximal end of electrode lead 107. The proximal end of electrode lead 107 connects to a pacemaker 106.

The electrode lead 107 is placed in the heart using standard cardiac lead placement devices, including introducers, guide catheters, guide wires, and/or stylets. Briefly, an introducer is placed into the subclavian vein. A guide catheter is placed through the introducer and used to locate the entrance to the coronary sinus in the right atrium. A guide wire is then used to locate a left ventricular cardiac vein. The electrode lead 107 is slid over the guide wire into the left ventricular cardiac vein 104 and tested until an optimal location for CRT is found. 5 Once implanted, a multi-electrode lead 107 still allows for continuous readjustments of the optimal electrode location.

The electrode lead 109 is placed in the right ventricle of the heart with an active fixation helix at the distal end 119, which is embedded into the cardiac septum. The electrode lead 109 is provided with one or multiple electrodes 113, 114 and 115. The distal tip of the electrode lead 109 is provided with an active fixation helix 116, which is screwed into the mid-septum 103.

Electrode lead 109 is placed in the heart using standard cardiac lead devices, including introducers, guide catheters, guide wires, and/or stylets. Electrode lead 109 is inserted into the subclavian vein, through the superior vena cava, through the right atrium, and down into the right ventricle. Electrode lead 109 is positioned under fluoroscopy into the location determined to be clinically optimal and logistically practical for fixating the electrode lead 109 and obtaining motion timing information for the cardiac feature area surrounding the attachment site. Under fluoroscopy, the active fixation helix 116 is advanced and screwed into the cardiac tissue to secure electrode lead 109 onto the septum. The electrode lead 109 is typically fabricated of a soft flexible material with the capacity to conform to the shape of the heart chamber.

Lead 108 is placed in the right atrium of the heart. The distal end 118 of lead 108 is attached to the inner cardiac wall using any suitable means. Lead 108 does not contain electrodes. The motion of the right atrium can be detected by determining the motion of contrast markers in lead 108 that appear in images generated by an external imaging device, according to an embodiment of the present invention.

Each of leads 107, 108 and 109 contains one or more contrast markers according to embodiments of the present invention. The contrast markers can be, for example, embedded into the body of the leads during the construction of the leads or crimped onto the outside of the leads. Leads 107, 108 and 109 are merely three examples of leads that can contain contrast markers according to the present invention. Contrast markers can be included in any type of lead that is implanted inside or outside a heart, including leads with or without electrodes.

The contrast markers may be implanted near the distal ends 117, 118 and 119, respectively, of leads 107, 108 and 109 where the distal ends of the leads come into contact with the cardiac tissue. For example, the markers can be placed on or near the septum, the left ventricular free wall, and the right atrium wall of the heart. Alternatively, the contrast markers can be placed in the leads far away from the distal ends to monitor other regions of the heart. In another embodiment, the contrast marker can be a stand-alone structure stably associated with a tissue location.

The contrast markers are any object or device that can be position-located using an external imaging device. Examples of contrast markers that can be implanted into leads 107, 108 and 109 include ultrasound reflectors, such as air-filled spheres, metal balls, wires, retro-reflectors, etc. Ultrasonic signals usually reflect off of most hard surfaces. An ultrasound reflector can be made of any material that does not absorb sound signals. Ultrasonic signals can also reflect off of the boundaries between two different materials. As long as the contrast markers have a sufficiently large difference in acoustic impedance relative to the heart tissue, they will be clearly visible in images generated by an ultrasound modality.

A contrast marker can be any type of object that absorbs, emits, or reflects energy and, as a result, is detectable in images generated by an external imaging device. For example, a contrast marker can be a radio-opaque marker. As yet another example, a contrast marker can include a liquid contrast medium that is embedded in small spaces within a lead. According to some embodiments of the present invention, a contrast marker can provide contrast without absorbing or reflecting, e.g.,a fatty material or gadolinium in a magnetic resonance imaging (MRI) scan.

After the leads are implanted in the heart, the contrast markers can be located by analyzing images (e.g., video) generated by a suitable imaging device, e.g., an internal or external imaging device. Cardiac motion can be calculated based on the positions of the contrast markers and how they change from a first image to a second image produced using an internal or external imaging device.

Examples of imaging technology that can be used by an external imaging device to locate the contrast markers include ultrasound/sonography (A-mode or B-mode), fluoroscopy, X-ray radiographs, magnetic resonance imaging (MRI), three-dimensional computer tomography (CT) scans or computed axial tomography (CAT) scans, infrared tomography, nuclear medicine, elastography, electrical impedance tomography, optoacoustic imaging, positron emission tomography (PET) scans, and other types of imaging devices generating signals that can penetrate tissue. The imaging device can be an ultrasound device, a fluoroscopy device, or an MRI device. Images generated from nuclear medicine are developed based on the detection of energy emitted from a radioactive substance given to the patient, e.g., intravenously or by mouth.

According to further embodiments of the present invention, the contrast markers can be oriented in particular ways that allow them to reflect, emit, or absorb more electromagnetic radiation, ultrasonic waves, or other signals to increase the contrast between the markers and the surrounding tissue. Particular orientations of the contrast markers can allow them to be more accurately and more precisely detected in images generated by an external imaging device. For example, reflective surfaces of reflective markers can be oriented toward the chest of a patient to increase reflection from an external imaging device located on the chest.

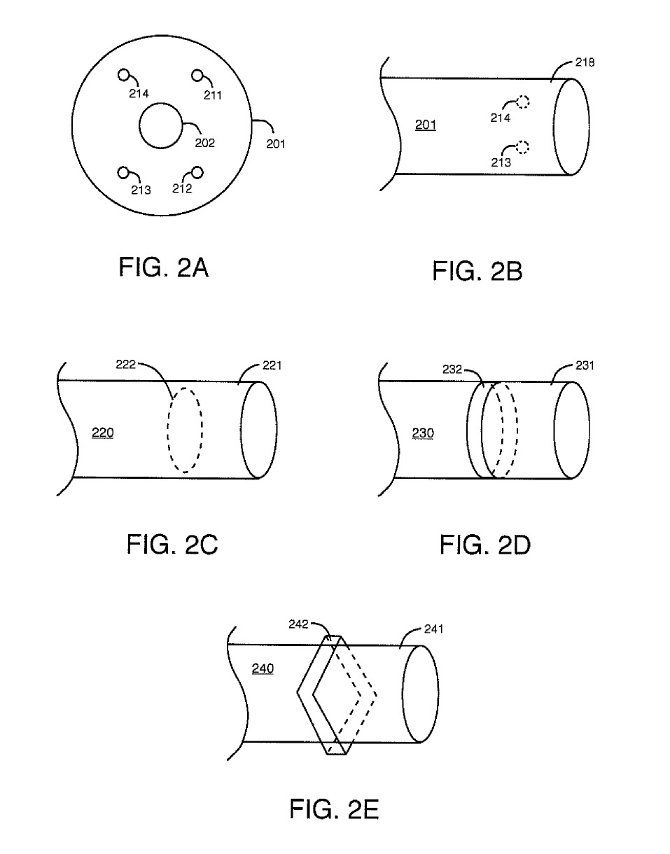

FIGS. 2A to 2B illustrate contrast markers that are embedded within leads of a tomography device, according to an embodiment of the present invention. The tomography device can include an implantable pulse generator, e.g., configured as a pacemaker, or any other device that senses or regulates heart motion. FIG. 2A shows a cross-sectional view of the distal end of a lead 201 that is connected to a tomography device. Lead 201 includes a cylindrical inner cavity 202. A guide wire is inserted into cavity 202 so that lead 201 can be guided into a desired location within a patient.

Lead 201 also includes contrast markers 211-214 that are visible in images generated by an external imaging device, according to an embodiment of the present invention. Contrast markers 211-214 are embedded into the body of lead 201. Markers 211-214 can be placed in a mold as lead 201 is formed so that markers 211-214 are embedded inside the body of the lead during the molding process. Lead 201 can be made of silicone or polyurethane, for example. Markers 211-214 can absorb, emit, or reflect external signals that penetrate tissue, such as ultrasound waves, magnetic fields, or X-rays.

FIG. 2B illustrates a longitudinal view of lead 201 that contains embedded contrast markers 211-214. Markers 213 and 214 are visible in the longitudinal view of FIG. 2B. Markers 211-214 are embedded in lead 201 near its distal end 218.

FIG. 2C illustrates a longitudinal view of a lead 220 that contains a circular wire 222. Circular wire 222 is embedded in the body of lead 220 near its distal end 221. Circular wire 222 is a contrast marker that is visible in images generated by an external imaging device, according to another embodiment of the present invention. Circular wire 222 can absorb, reflect, or emit any type of signal that can penetrate tissue and that can be emitted by an external imaging device.

FIG. 2D illustrates a longitudinal view of a lead 230 that contains a cylindrical contrast marker 232 near its distal end 231. Marker 232 is visible in images generated by an external imaging device, according to another embodiment of the present invention. Marker 232 is formed around the outside of lead 230. For example, marker 232 can be a metal that is plated around the outside of lead 230. Marker 232 contains a material that absorbs, reflects, or emits any type of signal that can penetrate tissue and that can be generated by an external imaging device.

FIG. 2E illustrates a longitudinal view of a lead 240 that contains a contrast marker 242 near its distal end 241. Marker 242 is crimped around the outside of lead 240. Marker 242 can comprise, for example, a flexible metal. Marker 242 is visible in images generated by an external imaging device, according to another embodiment of the present invention. Marker 242 contains a material that absorbs, emits, or reflects any type of signal that can penetrate tissue and that can be generated by an external imaging device.

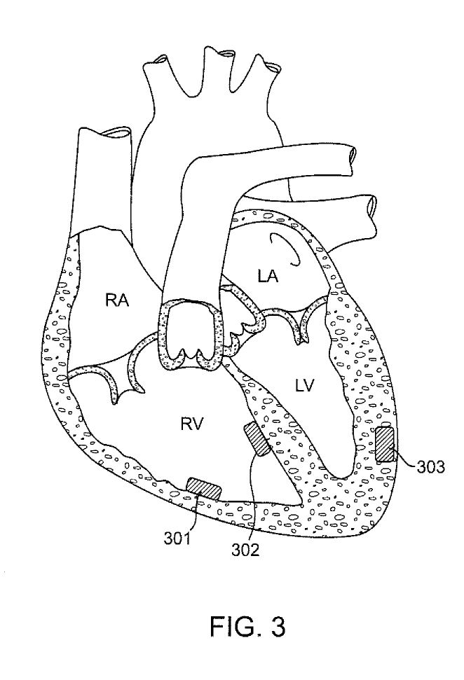

FIG. 3 illustrates stand-alone contrast markers 301-303 that are attached to cardiac tissue according to another embodiment of the present invention. Marker 301 is attached to right ventricle wall, marker 302 is attached to the cardiac septum, and marker 303 is located in the left ventricular cardiac vein. Contrast markers 301-303 can contain any type of material that can be identified in images generated by an external imaging device. For example, markers 301-303 can contain embedded objects such as air-filled spheres, metal balls, wires, or retro-reflectors that have a large acoustic impedance relative to the cardiac tissue so that they can be detected using an ultrasound imaging device. As another example, contrast markers 301-303 can be objects that absorb, emit, or reflect X-rays, magnetic fields, or another type of radiation.

Stand-alone contrast markers 301-303 can be introduced into the heart in any suitable fashion. For example, markers 301-303 can be inserted into the heart using a catheter or directly injected into the pericardial sac of the heart. The contrast markers can be attached to the cardiac tissue using any suitable means. For example, the markers can be screwed into the cardiac tissue or attached using a fixation helix.

According to another embodiment of the present invention, a liquid contrast marker is injected into the heart and absorbed by the cardiac tissue. The liquid contrast marker responds differently to signals from an external imaging device than the surrounding tissue. For example, the liquid can contain barium, which reflects X-rays. A liquid marker can allow a temporary determination and analysis of cardiac motion.

According to yet another embodiment of the present invention, the contrast markers can be very small nano-markers that are detectable in images generated by an external imaging device. The nano-markers can be implanted directly into the heart or into the leads of a tomography device.

Just a few examples of contrast markers that can be used to implement the techniques of the present invention are discussed herein. It should be understood that the present invention applies to any type of contrast marker, permanent or temporary, which can be detected in images generated by an external imaging device.

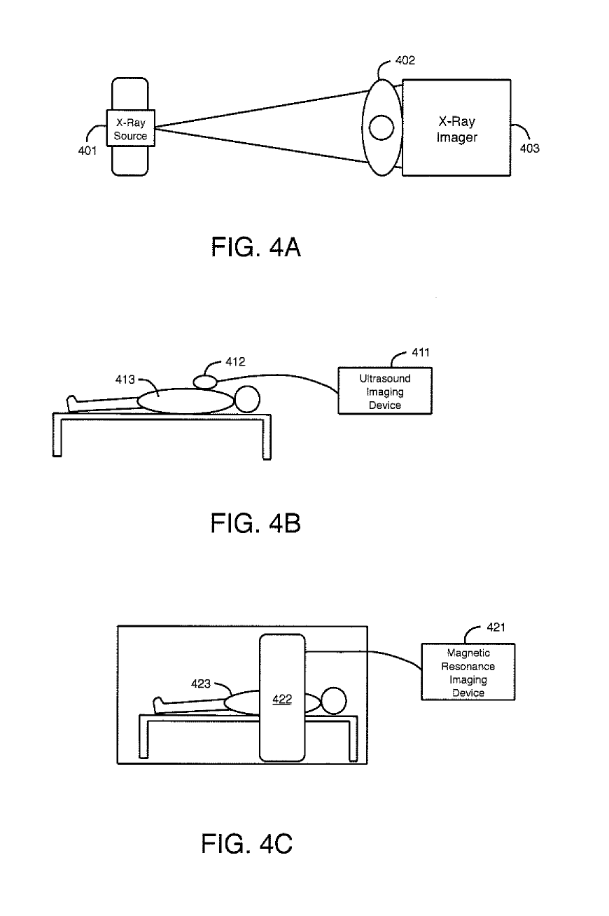

FIGS. 4A to 4C illustrate three types of external imaging devices that can generate images of contrast markers in cardiac tissue, according to various embodiments of the present invention. FIG. 4A illustrates a top-down view of a fluoroscopy system that can be used to locate cardiac contrast markers. The fluoroscopy system includes an X-ray source 401 that generates X-rays. The X-rays from source 401 pass through patient 402 and are detected by X-ray imager 403.

FIG. 4B illustrates a side-view of an ultrasound system that can generate images of cardiac contrast markers. Ultrasound generator and sensor 412 is placed on the chest of patient 413 above the heart region. Generator and sensor 412 generates ultrasound waves and senses ultrasound waves that are reflected back to the sensor, according to well-known techniques. Signals representing the reflected wave patterns are transmitted from sensor 412 to ultrasound imaging device 411 for display, analysis, and processing.

FIG. 4C illustrates a side-view of a magnetic resonance imaging (MRI) system that can be used to locate cardiac contrast markers. A patient 423 is placed inside a MRI generator and sensor 422 as shown in FIG. 4C. MRI 422 is typically shaped like a donut. MRI 422 generates and senses magnetic fields according to well-known techniques. MRI 422 transmits output signals to magnetic resonance imaging device 421 for display, analysis, and processing.

As summarized above, the present invention includes systems and methods for identifying contrast markers in images generated by an external imaging device in order to objectively quantify cardiac motion for the purpose of providing cardiac therapies, such as resynchronization. Embodiments of the methods include determining motion of a tissue location in a subject by: (a) locating a first contrast marker in a first image, wherein the first contrast marker is stably associated with said tissue location of interest; (b) locating said first contrast marker in a second image that is taken at a time point after the first image, and (c) evaluating motion of said the contrast marker in the first image relative to the first contrast marker in the second image to determine motion of the tissue location. In certain embodiments, the first image is located in multiple time sequential images and motion evaluated automatically. In certain embodiments, the methods further include locating a second contrast marker in the first and second images; and evaluating motion of said first contrast marker relative to the second contrast marker in the second image as compared to the first image. In certain embodiments, after locating the first and second contrast markers, their motion relative to each other in the images is evaluated automatically.

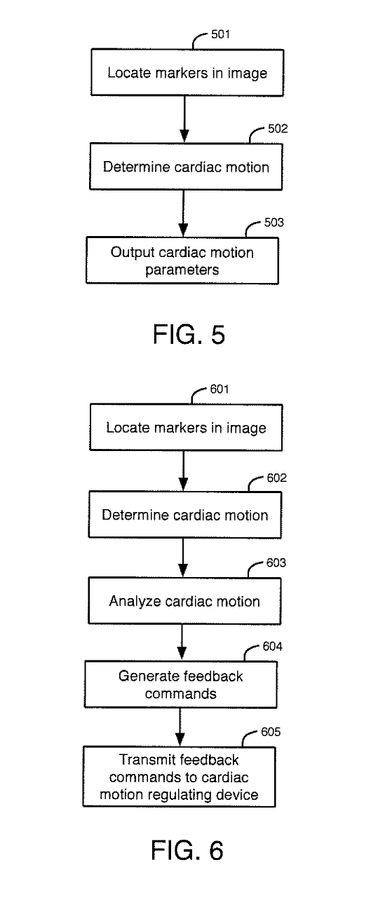

FIG. 5 illustrates an example of a process for locating contrast markers in images generated by an external imaging device and outputting quantifiable data regarding cardiac motion, according to an embodiment of the present invention.

At step 501, an analysis is performed of images of a heart that are generated by an imaging device, such as an external imaging device. The images generated by the imaging device contain indications of the contrast markers located in the heart. However, the images are usually very fuzzy, and therefore, it can be difficult for a clinician to locate contrast markers in the images. Often, clinicians cannot locate contrast markers in a way that is repeatable and reproducible from session to session merely by looking at pre-processed images. A significant amount of subjectivity and error is introducing into the process of identifying the contrast markers using only unaided human eyes.

According to an embodiment of the present invention, real-time image recognition software is used to identify the location of the cardiac contrast markers in images generated by an external imaging device. The real-time image recognition software identifies the location of the markers in two or more images, e.g., a first and second image, by analyzing the contrast between the markers and the surrounding tissue at step 501. Any convenient real-time image recognition software, such as software that can provide image recognition, feature extraction, and image quantification may be employed.

Contrast markers can, for example, be equipped with unique features that allow each marker to be separately identified in a set of images. For example, the markers can have different shapes and sizes, different reflection properties, different emission properties, or different absorption properties. The different properties of each contrast marker can allow the image recognition software to more easily identify each individual marker in the images.

At step 502, cardiac motion is determined based on the motion of the contrast markers in the images. The relative motion of the contrast markers in the images are compared to determine the relative motion of various regions of the heart. For example, the motion of the septum relative to the left ventricle free wall can be determined based on the relative motion of one marker attached to the septum and a second marker attached to the left ventricle free wall. From evaluation of this data, a motion value for the tissue location (e.g. cardiac) can be determined. This motion value can be compared to a reference value to generate data that is useful for diagnosis and follow-up treatments.

At step 503, cardiac motion output parameters are generated based on the relative motion of the contrast markers. The output parameters can be provided in a dynamic real-time mode or in a static display for a user or to another device. The output parameters can include raw data, such as position data, time data, and/or motion data regarding each region of the heart that is tracked by a contrast marker. As another example, the cardiac motion output parameters can include a set of waveforms or a series of traces, where each waveform or trace depicts the motion of one or more of the contrast markers. As another example, the cardiac motion output parameters can be provided as time delay output data in text or graphical format that indicates delays between the contractions of various regions of the heart.

The cardiac output parameters can also include data that is manipulated based on a set of input parameters. The input parameters can include, for example, data indicating that the interventricular septum and the left ventricular free wall are supposed to contract at approximately the same time. The input parameters can also indicate a degree deviation from exact synchronous contraction that is considered acceptable, e.g., as may be generated by comparing to a reference value. The relative motion of the regions of the heart that are tracked by contrast markers can be compared to the input parameters to generate output data that is useful for diagnosis and for providing follow-up treatments. For example, the relative contraction times of the interventricular septum and the left ventricular free wall that are determined at step 502 can be compared to the input parameters to generate an output that indicates a deviation of the relative contraction times from preferred values.

FIG. 6 illustrates an example of a process for analyzing cardiac motion using contrast markers and producing feedback commands that control the regulation of cardiac motion, according to another embodiment of the present invention, where the feedback commands are based on the determined motion of the tissue location, i.e., produced at least in part on the determined motion of the tissue location. At step 601, the contrast markers in images (e.g., at least a first and second image) generated by an external imaging device are located using, for example, image recognition software. At step 602, cardiac motion is inferred by determining the motion of the contrast markers in the images, as described above.

At step 603, the cardiac motion is analyzed by comparing the location and/or the motion of the contrast markers to other parameters, e.g., reference values, etc. For example, data indicating the movement of the markers can be compared to input data (or stored data) that indicates ideal values for cardiac motion. The input data can be entered manually by a clinician or generated from a monitor such as an electrocardiogram (ECG). An ECG can perform diagnostic tests that analyze the electrical activity of the heart, including the heartbeat. As another example, the data indicating the motion of the markers can be compared to input scripts that tend to be efficacious for particular categories of patients.

The present invention can generate real-time data that indicates a deviation between the measured values of cardiac motion and ideal values. For example, the synchronization and contractility of various regions of the heart can be compared to ideal synchronization and contractility values to compute the deviation.

At step 604, feedback commands are generated based on the deviations computed at step 603. The feedback commands are forwarded to an implantable pulse generator, such as a cardiac motion regulating device (e.g. a pacemaker). The feedback commands can be used to operate the cardiac motion regulating device, e.g., in the form of an implantable pulse generator, to adjust the motion of the heart to reduce or eliminate the deviation of the heart motion relative to the ideal values. For example, the feedback commands can cause a pacemaker to perform cardiac resynchronization therapy to induce the interventricular septum and the left ventricular free wall to contract at approximately the same time by adjusting the pacemaker's IVD and/or atrio-ventricular delay settings. CRT optimization can be used to reduce the number of permutations of an algorithm that need to be performed to optimize the cardiac synchrony.

At step 605, the feedback commands are transmitted to the cardiac motion regulating device to provide a real-time cardiac motion regulatory control system. For example, if the cardiac motion device is a pacemaker that is implanted into the chest of a patient, the feedback commands can be transmitted wirelessly to the pacemaker, e.g., using radio frequency (RF) signals or magnetic/electrical fields generated from two inductors that form a transformer when placed close together. Alternatively, the feedback commands can be forwarded to the cardiac motion device through a wired connection. The cardiac motion regulating device can then be operated in response to the forwarded feedback command, e.g., to achieve CRT.

An image recognition software product can be used to help identify contrast markers in images generated by an external imaging device. For example, commercially available image recognition software products are designed by Matrox Graphics Inc. of Quebec, Canada, and National Instruments Corp. of Austin, Tex.

A specific embodiment is now described regarding how a software image recognition product can be used to locate contrast markers in images of cardiac tissue. This specific embodiment is described for illustrative purposes only and is not intended to limit the scope of the present invention in any way.

Still images (i.e., frames) of heart tissue generated by an external imaging device can be combined into a video file (e.g., an .avi file). A series of functions are then performed on the video file to increase the contrast between fiducials in the images and the background. The fiducials are dark regions in the images that could potentially be images of contrast markers. Initially, the frames are pre-processed using an open filter that smoothes out dots in the background. Then, the frames are processed again using a sharpening filter that is based on a Deriche function. Once the frames have been processed, the fiducials are darker with respect to the background.

Each pixel in a non-color frame is represented by a grayscale number between 1 and 256, where 1 is black, and 256 is white. The first frame is binarized by setting each pixel that is below a threshold grayscale value to black (1). Each pixel that is above or equal to the threshold grayscale value is set to white (256). The result is a binarized black and white image.

Each black region in the binarized frame is labeled with a symbol and/or a number. A user then manually selects one or more of the black regions in the frame that he wants to track after deciding which of the regions appear to be images of the contrast markers. Analysis of the full video file can then begin.

Each black region in the first frame of the video is defined by a center point of the region and a region size (i.e., based on its height and width). The center point of each black region is the starting point for the next frame. The image recognition software looks for each black region in the next frame within the region area with respect to the center point of that region in the previous frame. If the black region moves outside the region area between two frames, the image recognition software loses track of that region. The height and width of a black region can be increased to keep track of rapidly moving regions. In each subsequent frame, the center point of the region is reset based on the location of the region in the current frame. Preferably, any dark areas that appear on the border of a region are excluded.

Each subsequent frame is binarized using the threshold grayscale value. Each black region can have a different threshold grayscale value that is based on the darkest pixel in that region. The threshold grayscale value is added to the grayscale value of the darkest pixel in each black region to generate the threshold value for that region. The threshold value for each black region is used to binarize that region in every frame of the video.

Additional filters can be run to prevent the tracking of certain regions in the video file that are not likely to be contrast markers. For example, minimum and maximum area filters can be used to exclude (e.g., white-out) dark regions that are below a minimum size and above a maximum size. The minimum and maximum values can be adjusted by the user based on information about the sizes of the contrast markers. A compactness filter can be used to eliminate dark regions having shapes that make them unlikely to be contrast markers. A circle has the lowest compactness value, and as the shape of a region deviates from a circle, its compactness value increases. A user can select a range of compactness values to prevent the tracking of regions that are outside the range.

The processes described with respect to FIGS. 5-7 can be implemented by any suitable computer system. For example, the process of FIG. 5 can be implemented on a laptop or a desktop computer that is connected to receive imaging data from an external imaging device such as an ultrasound machine, an MRI, or a fluoroscopy device. As another example, the process of FIG. 6 can be implemented on a laptop or a desktop computer connected to an external imaging device and a device that can reprogram an implanted pacemaker device when a patient visits a physician's office.

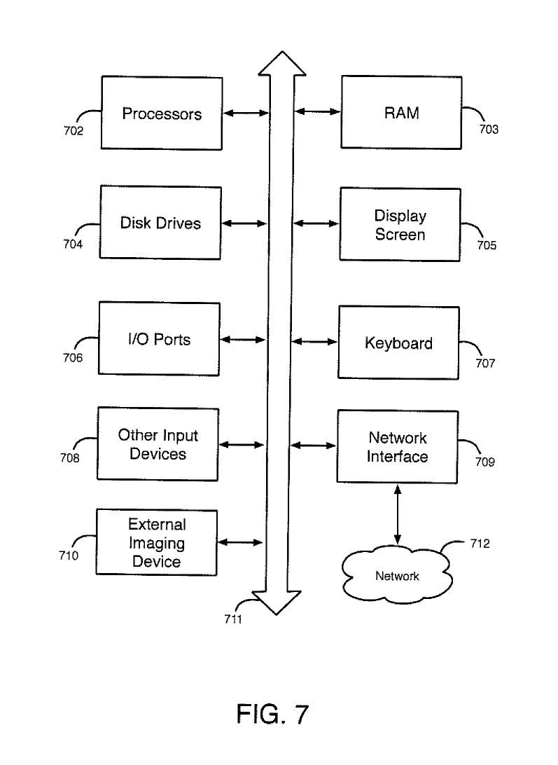

FIG. 7 illustrates an example of a computer system 700 that is capable of implementing embodiments of the present invention. Computer system 700 typically includes components such as one or more general purpose processors 702, memory storage devices such as a random access memory (RAM) 703 and disk drives 704, a display screen 705, input/output (I/O) ports 706, and a system bus 711 that interconnects these components and other components. The memory storage devices can store data, graphics, and code that is used according to embodiments of the present invention.

Display screen 705 can display output data such as waveforms and raw data according to embodiments of the present invention. Processors 702 can run code to implement methods of the present invention, such as the processes described above with respect to FIGS. 5 and 6. I/O ports 706 are interfaces that allow computer system 700 to communicate with heart motion regulating devices, such as pacemakers.

Computer system 700 can communicate with additional devices such as a keyboard 707, other input devices 708, a network interface 709, and an external imaging device 710. Other input devices 708 can include, for example, a computer mouse, a trackball, a touch screen, and/or other wired or wireless input devices, Network interface 709 typically provides wired or wireless communication with a communications network 712. Network 712 can be, for example, a local area network, a wide area network (e.g., the Internet), or a virtual network. If desired, computer system 700 can communicate with external imaging device 710 through network 712 and network interface 709, instead of through system bus 711.

Systems

Aspects of the invention include systems, including implantable medical devices and systems, which include the devices of the invention and can be employed to practice methods according to the invention, e.g., as described above. The systems may also be configured to perform a number of different functions, including but not limited to electrical stimulation applications, e.g., for medical purposes, such as pacing, CRT, etc.

The systems for determining motion of a tissue location in a subject may have a number of different components or elements. Elements that are present in the systems may include an imaging device, contrast markers, and a signal processing element configured to perform the methods outlined above, e.g., for implementing the protocol depicted in FIGS. 5 and 6.

In certain embodiments of the subject systems, one or more contrast markers of the invention are present as stand-alone contrast markers. In certain embodiments of the subject systems, one or more receive contrast markers of the invention are present on at least one elongated conductive member, e.g., an elongated conductive member present in a lead, such as a cardiovascular lead. In certain embodiments of the subject systems, two or more contrast markers of the invention are present as stand-alone contrast markers or are present on at least one elongated conductive member, e.g., an elongated conductive member present in a lead, such as a cardiovascular lead. In certain embodiments, the elongated conductive member is part of a multiplex lead, e.g., as described in Published PCT Application No. WO 2004/052182 and U.S. patent application Ser. No. 10/734,490, the disclosure of which is herein incorporated by reference. In some embodiments of the invention, the devices and systems may include onboard logic circuitry or a processor, e.g., present in a central control unit, such as a pacemaker can. In these embodiments, the central control unit may be electrically coupled to one or more receive electrodes via one or more conductive members.

In certain embodiments of the subject systems, one or more sets of electrodes are electrically coupled to at least one elongated conductive member, e.g., an elongated conductive member present in a lead, such as a cardiovascular lead. In certain embodiments, the elongated conductive member is part of a multiplex lead. Multiplex lead structures may include 2 or more satellites, such as 3 or more, 4 or more, 5 or more, 10 or more, 15 or more, 20 or more, etc. as desired, where in certain embodiments multiplex leads have a fewer number of conductive members than satellites. In certain embodiments, the multiplex leads include 3 or less wires, such as only 2 wires or only 1 wire. Multiplex lead structures of interest include those described in application Ser. No. 10/734,490 titled “Method and System for Monitoring and Treating Hemodynamic Parameters” filed on Dec. 11, 2003; PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; 60/793,295 titled “High Phrenic, Low Pacing Capture Threshold Implantable Addressable Segmented Electrodes” filed on Apr. 18, 2006 and 60/807,289 titled “High Phrenic, Low Capture Threshold Pacing Devices and Methods,” filed Jul. 13, 2006; the disclosures of the various multiplex lead structures of these applications being herein incorporated by reference. In some embodiments of the invention, the devices and systems may include onboard logic circuitry or a processor, e.g., present in a central control unit, such as a pacemaker can. In these embodiments, the central control unit may be electrically coupled to the lead by a connector, such as a proximal end IS-1 connection.

In certain embodiments, the leads are characterized by the presence of segmented electrode structures. By segmented electrode structure is meant an electrode structure that includes two or more, e.g., three or more, including four or more, disparate electrode elements. Embodiments of segmented electrode structures are disclosed in Application Serial Nos.: PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; 60/793,295 titled “High Phrenic, Low Pacing Capture Threshold Implantable Addressable Segmented Electrodes” filed on Apr. 18, 2006 and 60/807,289 titled “High Phrenic, Low Capture Threshold Pacing Devices and Methods,” filed Jul. 13, 2006; the disclosures of the various segmented electrode structures of these applications being herein incorporated by reference.

In certain embodiments, the leads are characterized by the presence of electrodes that are “addressable” electrode structures. Addressable electrode structures include structures having one or more electrode elements directly coupled to control circuitry, e.g., present on an integrated circuit (IC). Addressable electrode structures include satellite structures that include one more electrode elements directly coupled to an IC and configured to be placed along a lead. Examples of addressable electrode structures that include an IC are disclosed in application Ser. No. 10/734,490 titled “Method and System for Monitoring and Treating Hemodynamic Parameters” filed on Dec. 11, 2003; PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; 60/793,295 titled “High Phrenic, Low Pacing Capture Threshold Implantable Addressable Segmented Electrodes” filed on Apr. 18, 2006 and 60/807,289 titled “High Phrenic, Low Capture Threshold Pacing Devices and Methods,” filed Jul. 13, 2006; the disclosures of the various addressable electrode structures of these applications being herein incorporated by reference.

Embodiments of the subjects systems may incorporate one or more effector elements. The effectors may be intended for collecting data, such as but not limited to pressure data, volume data, dimension data, temperature data, oxygen or carbon dioxide concentration data, hematocrit data, electrical conductivity data, electrical potential data, pH data, chemical data, blood flow rate data, thermal conductivity data, optical property data, cross-sectional area data, viscosity data, radiation data and the like. As such, the effectors may be sensors, e.g., temperature sensors, accelerometers, ultrasound transmitters or receivers, AC voltage sensors, potential sensors, current sensors, etc. Alternatively, the effectors may be intended for actuation or intervention, such as providing an electrical current or voltage, setting an electrical potential, heating a substance or area, inducing a pressure change, releasing or capturing a material or substance, emitting light, emitting sonic or ultrasound energy, emitting radiation and the like.

Effectors of interest include, but are not limited to, those effectors described in the following applications by at least some of the inventors of the present application: U.S. patent application Ser. No. 10/734,490 published as 20040193021 titled: “Method And System For Monitoring And Treating Hemodynamic Parameters”; U.S. patent application Ser. No. 11/219,305 published as 20060058588 titled: “Methods And Apparatus For Tissue Activation And Monitoring”; International Application No. PCT/US2005/046815 titled: “Implantable Addressable Segmented Electrodes”; U.S. patent application Ser. No. 11/324,196 titled “Implantable Accelerometer-Based Cardiac Wall Position Detector”; U.S. patent application Ser. No. 10/764,429, entitled “Method and Apparatus for Enhancing Cardiac Pacing,” U.S. patent application Ser. No. 10/764,127, entitled “Methods and Systems for Measuring Cardiac Parameters,” U.S. patent application Ser. No. 10/764,125, entitled “Method and System for Remote Hemodynamic Monitoring”; International Application No. PCT/US2005/046815 titled: “Implantable Hermetically Sealed Structures”; U.S. application Ser. No. 11/368,259 titled: “Fiberoptic Tissue Motion Sensor”; International Application No. PCT/US2004/041430 titled: “Implantable Pressure Sensors”; U.S. patent application Ser. No. 11/249,152 entitled “Implantable Doppler Tomography System,” and claiming priority to: U.S. Provisional Patent Application No. 60/617,618; International Application Serial No. PCT/USUS05/39535 titled “Cardiac Motion Characterization by Strain Gauge”. These applications are incorporated in their entirety by reference herein.

Use of the systems may include visualization of data obtained with the devices. Some of the present inventors have developed a variety of display and software tools to coordinate multiple sources of sensor information which will be gathered by use of the inventive systems. Examples of these can be seen in international PCT application serial no. PCT/US2006/012246; the disclosure of which application, as well as the priority applications thereof are incorporated in their entirety by reference herein.

Data obtained in accordance with the invention, as desired, can be recorded by an implantable computer. Such data can be periodically uploaded to computer systems and computer networks, including the Internet, for automated or manual analysis. In one embodiment, the signal processing element used to process data according to the subject methods can comprise an image recognition algorithm.

Uplink and downlink telemetry capabilities may be provided in a given implantable system to enable communication with either a remotely located external medical device or a more proximal medical device on the patient's body or another multi-chamber monitor/therapy delivery system in the patient's body. The stored physiologic data of the types described above as well as real-time generated physiologic data and non-physiologic data can be transmitted by uplink RF telemetry from the system to the external programmer or other remote medical device in response to a downlink telemetry transmitted interrogation command. The real-time physiologic data typically includes real time sampled signal levels, e.g., intracardiac electrocardiogram amplitude values, and sensor output signals including dimension signals developed in accordance with the invention. The non-physiologic patient data includes currently programmed device operating modes and parameter values, battery condition, device ID, patient ID, implantation dates, device programming history, real time event markers, and the like. In the context of implantable pacemakers and ICDs, such patient data includes programmed sense amplifier sensitivity, pacing or cardioversion pulse amplitude, energy, and pulse width, pacing or cardioversion lead impedance, and accumulated statistics related to device performance, e.g., data related to detected arrhythmia episodes and applied therapies. The multi-chamber monitor/therapy delivery system thus develops a variety of such real-time or stored, physiologic or non-physiologic, data, and such developed data is collectively referred to herein as “patient data”.

Utility

The methods of evaluating tissue location movement find use in a variety of different applications. As indicated above, one application of the subject invention is for use in cardiac resynchronization therapy (CRT) (i.e., biventricular pacing). CRT remedies the delayed left ventricular mechanics of heart failure patients. In a desynchronized heart, the inter-ventricular septum will often contract ahead of portions of the free wall of the left ventricle. In such a situation, where the time course of ventricular contraction is prolonged, the aggregate amount of work performed by the left ventricle against the intra-ventricular pressure is substantial. However, the actual work delivered on the body in the form of stroke volume and effective cardiac output is lower than would otherwise be expected. Using the subject approach, the electromechanical delay of the left lateral ventricle can be evaluated and the resultant data employed in CRT, e.g., using the approaches reviewed above and/or known in the art and reviewed at Col. 22, lines 5 to Col. 24, line 34 ff of U.S. Pat. No. 6,795,732, the disclosure of which is herein incorporated by reference.

In a fully implantable system the location of the pacing electrodes on multi electrode leads and pacing timing parameters are continuously optimized by the pacemaker. The pacemaker frequently determines the location and parameters which minimizes intra-ventricular dyssynchrony, interventricular dyssynchrony, or electromechanical delay of the left ventricle lateral wall in order to optimize CRT. This cardiac wall motion sensing system can also be used during the placement procedure of the cardiac leads in order to optimize CRT. An external controller could be connected to the cardiac leads and a skin patch electrode during placement of the leads. The skin patch acts as the reference electrode until the pacemaker is connected to the leads. In this scenario, for example, the optimal left ventricle cardiac vein location for CRT is determined by acutely measuring intra-ventricular dyssynchrony.

The subject methods and devices can be used to adjust a resynchronization pacemaker either acutely in an open loop fashion or on a nearly continuous basis in a closed loop fashion.

Other uses for this system are as an ischemia detector. It is well understood that in the event of acute ischemic events one of the first indications of such ischemia is akinesis, i.e., decreased wall motion of the ischemic tissue as the muscle becomes stiffened. A Wall motion system would be a very sensitive indicator of an ischemic process, by ratio metrically comparing the local wall motion to a global parameter such as pressure; this has been previously described in another Proteus patent. One can derive important information about unmonitored wall segments and their potential ischemia. For example, if an unmonitored section became ischemic, the monitored segment would have to work harder and have relatively greater motion in order to maintain systemic pressure and therefore ratio metric analysis would reveal that fact.

Another application of such position indicators that record wall motion is as a superior arrhythmia detection circuit. Current arrhythmia detection circuits rely on electrical activity within the heart. Such algorithms are therefore susceptible to confusing electrical noise for an arrhythmia. There is also the potential for misidentifying or mischaracterizing arrhythmia based on electrical events when mechanical analysis would reveal a different underlying physiologic process. Therefore the current invention could also be adapted to develop a superior arrhythmia detection and categorization algorithm.

Additional applications in which the subject invention finds use include, but are not limited to: the detection of electromechanical dissociation during pacing or arrhythmias, differentiation of hemodynamically significant and insignificant ventricular tachycardias, monitoring of cardiac output, mechanical confirmation of capture or loss of capture for autocapture algorithms, optimization of multi-site pacing for heart failure, rate responsive pacing based on myocardial contractility, detection of syncope, detection or classification of atrial and ventricular tachyarrhythmias, automatic adjustment of sense amplifier sensitivity based on detection of mechanical events, determination of pacemaker mode switching, determining the need for fast and aggressive versus slower and less aggressive anti-tachyarrhythmia therapies, or determining the need to compensate for a weakly beating heart after therapy delivery (where these representative applications are reviewed in greater detail in U.S. Pat. No. 6,795,732, the disclosure of which is herein incorporated by reference), and the like.

In certain embodiments, the subject invention is employed to overcome barriers to advances in the pharmacologic management of CHF, which advances are slowed by the inability to physiologically stratify patients and individually evaluate response to variations in therapy. It is widely accepted that optimal medical therapy for CHF involves the simultaneous administration of several pharmacologic agents. Progress in adding new agents or adjusting the relative doses of existing agents is slowed by the need to rely solely on time-consuming and expensive long-term morbidity and mortality trials. In addition, the presumed homogeneity of clinical trial patient populations may often be erroneous since patients in similar symptomatic categories are often assumed to be physiologically similar. It is desirable to provide implantable systems designed to capture important cardiac performance and patient compliance data so that acute effects of medication regimen variation may be accurately quantified. This may lead to surrogate endpoints valuable in designing improved drug treatment regimens for eventual testing in longer-term randomized morbidity and mortality studies. In addition, quantitative hemodynamic analysis may permit better segregation of drug responders from non-responders thereby allowing therapies with promising effects to be detected, appropriately evaluated and eventually approved for marketing. The present invention allows for the above. In certain embodiments, the present invention is used in conjunction with the Pharma-informatics system, as described in PCT Application Serial No. PCT/US2006/016370 filed on Apr. 28, 2006; the disclosure of which is herein incorporated by reference.

Non-cardiac applications will be readily apparent to the skilled artisan, such as, by example, measuring the congestion in the lungs, determining how much fluid is in the brain, assessing distention of the urinary bladder. Other applications also include assessing variable characteristics of many organs of the body such as the stomach. In that case, after someone has taken a meal, the present invention allows measurement of the stomach to determine that this has occurred. Because of the inherently numeric nature of the data from the present invention, these patients can be automatically stimulated to stop eating, in the case of overeating, or encouraged to eat, in the case of anorexia. The present inventive system can also be employed to measure the fluid fill of a patient's legs to assess edema, or other various clinical applications.

Computer Readable Medium

One or more aspects of the subject invention may be in the form of computer readable storage media having a processing program stored thereon for implementing the subject methods. The computer readable storage media may be, for example, in the form of a computer disk or CD, a floppy disc, a magnetic “hard card”, a server, or any other computer readable media capable of containing data or the like, stored electronically, magnetically, optically or by other means. Accordingly, the processing program embodying steps for carrying-out the subject methods may be transferred or communicated to a processor, e.g., by using a computer network, server, or other interface connection, e.g., the Internet, or other relay means.

More specifically, a processor with a computer readable storage medium may include stored programming embodying an algorithm for carrying out the subject methods. Accordingly, such a stored algorithm is configured to, or is otherwise capable of, practicing the subject methods, e.g., by operating an implantable medical device to perform the subject methods. The subject algorithm and associated processor may also be capable of implementing the appropriate adjustment(s).

Of particular interest in certain embodiments are systems loaded with such computer readable mediums such that the systems are configured to practice the subject methods. For example, imaging devices loaded with programming on a computer readable storage medium that can implement the methods, e.g., as reviewed above, are provided.

Kits

As summarized above, also provided are kits for use in practicing the subject methods. The kits at least include a computer readable storage medium, as described above. The computer readable storage medium may be a component of other devices or systems, or components thereof, in the kit, such as an adaptor module, a contrast marker, a pacemaker, etc. The kits and systems may also include a number of optional components that find use, including but not limited to, implantation devices, etc.

In certain embodiments of the subject kits, the kits will further include instructions for using the subject devices or elements for obtaining the same (e.g., a website URL directing the user to a webpage which provides the instructions), where these instructions are typically printed on a substrate, which substrate may be one or more of: a package insert, the packaging, reagent containers and the like. In the subject kits, the one or more components are present in the same or different containers, as may be convenient or desirable.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

1. A method for determining motion of a tissue location in a subject, said method comprising:
(a) locating a first contrast marker in a first image, wherein said first contrast marker is stably associated with said tissue location;
(b) locating said first contrast marker in a second image that is taken at a time point after said first image; and
(c) evaluating motion of said first contrast marker in said first image relative to said first contrast marker in said second image to determine motion of said tissue location.

2. The method according to claim 1, wherein said method further comprises:
locating a second contrast marker in said first and second images; and
evaluating motion of said first contrast marker relative to said second contrast marker in said second image as compared to said first image.

3. The method according to claim 1, wherein said evaluating is performed automatically.

4. The method of claim 1, wherein said tissue location is a cardiac location.

5. The method according to claim 1, wherein said method is a method of determining cardiac wall motion.

6. The method according to claim 5, wherein said method is a method of detecting ventricular dyssynchrony.

7. The method according to claim 1, wherein said evaluating comprises producing a motion value for said tissue location.

8. The method according to claim 7, wherein said method further comprises comparing said motion value with a reference value.

9. The method according to claim 1, wherein said method further comprises generating an output parameter.

10. The method according to claim 9, wherein said output parameter is chosen from the group consisting of position data, time data and motion data.

11. The method according to claim 9, wherein said output parameter is provided in real-time mode.

12. The method according to claim 9, wherein said output parameter is provided In a static display.

13. The method according to claim 1, wherein said method further comprises producing a feedback command based on determined motion of said tissue location.

14. The method according to claim 13, wherein said method further comprises forwarding said feedback command to an implantable pulse generator.

15. The method according to claim 14, wherein said method further comprises operating said implantable pulse generator in response to said forwarded feedback command.

16. The method according to claim 15, wherein said method is a method of performing cardiac resynchronization therapy.

17. The method according to claim 1, wherein said first and second images are produced using an external imaging device.

18. The method according to claim 17, wherein said external imaging device is an ultrasound device.

19. The method according to claim 18, wherein said contrast marker comprises air-filled spheres, metal balls, wires or retro-reflectors.

20.-23. (canceled)

24. The method according to claim 1, wherein said contrast marker is present on a lead.

25. The method according to claim 1, wherein said contrast marker is a stand-alone structure stably associated with a tissue location.

26. A system for determining motion of a tissue location in a subject, said system comprising:
(a) an imaging device;
(b) a contrast marker; and
(c) a signal processing element configured to perform the method of claim 1.

27.-34. (canceled)

35. A computer readable storage medium having a processing program stored thereon, wherein said processing program operates a processor to perform a method according to claim 1.

36.-38. (canceled)
